(19) United States
(12) Patent Application Publication
(10) Pub. No.: US 2007/0112294 A1
(43) Pub. Date: May 17, 2007

(54) Iontophoresis device

(75) Inventors: Hidero Akiyama, Shibuya-ku (JP); Kiyoshi Kanamura, Shibuya-ku (JP); Mizuo Nakayama, Shibuya-ku (JP); Takehiko Matsumura, Shibuya-ku (JP); Akihiko Matsumura, Ohota-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Shibuya-ku (JP)

(21) Appl. No.: 11/522,496

(22) Filed: Sep. 14, 2006

Related U.S. Application Data
(60) Provisional application No. 60/726,803, filed on Oct. 14, 2005.

(30) Foreign Application Priority Data
Sep. 14, 2005 (JP) 2005-266623

(51) Int. Cl. A61N 1/30

(52) U.S. Cl. 604/20

(57) ABSTRACT

An iontophoresis device may be capable of preventing the generation of gas or ions upon energization, and/or may be capable of preventing the alteration of active agent ions due to an electrode reaction. Energization from an electrode to an active agent reservoir may be performed through an ionic liquid. The ionic liquid may include an anion such as PF6-, BF4-, AlCl4-, ClO4-, a hydrogen sulfate ion, bis-trifluoro-alkyl-sulfonyl-imide, or trifluoro-methane-sulfonate, and a cation such as an imidazolium derivative, a pyridinium derivative, a piperidinium derivative, a pyrolidinium derivative, and a tetra-alkyl-ammonium derivative.

### CROSS-REFERENCE TO RELATED APPLICATION

This application claims the benefit under 35 U.S.C. §119(e) of U.S. Provisional Patent Application No. 60/726,803, filed Oct. 14, 2005, now pending, which application is incorporated herein by reference in its entirety.

### BACKGROUND

1. Field

The present disclosure generally relates to the field of iontophoresis, and in particular, to an iontophoresis device capable of preventing or suppressing an electrode reaction in an electrode assembly.

2. Description of the Related Art

Iontophoresis involves using an electric potential to transdermally drive dissociated active agent ions in solution through a biological interface of a subject, transferring the active agent into the subject. Iontophoresis may reduce the burden placed on the subject when receiving the active agent, and may also allow for enhanced controllability.

FIG. 5 is an explanatory view that shows an iontophoresis device configuration.

The iontophoresis device of FIG. 5 comprises: an active electrode assembly 110 that includes an electrode 111 and an active agent solution reservoir 114 that holds a solution of an active agent which dissociates into positive or negative active agent ions (active agent solution); a counter electrode assembly 120 including an electrode 121 and an electrolyte solution reservoir 122 that holds an electrolyte solution; and an electric power source 130 that includes two terminals connected to the electrodes 111 and 121, respectively. An electric potential having the same polarity as that of active agent ions is applied to the electrode 111. An electric potential having a polarity opposite to that of the active agent ions is applied to the electrode 121 in a state where the active agent solution reservoir 114 and the electrolyte solution reservoir 122 are brought into contact with a biological interface of a subject. As a result, the active agent ions are administered to the subject.

In the iontophoresis device, electrode reactions may occur in the electrode assemblies 110 and 120.

For example, when a cationic active agent that dissociates into positive active agent ions is used, hydrogen ions or oxygen gas may be generated at the electrode 111 and hydroxide ions or hydrogen gas may be generated at the electrode 121 by the electrolysis of water. In addition, active agent ions may be altered due to an electrode reaction depending on the type of active agent used. Further, if the active agent solution reservoir 114 contains chlorine ions, chlorine gas or hypochlorous acid may be generated.

Similarly, when an anionic active agent that dissociates into negative active agent ions is used, hydroxide ions or hydrogen gas may be generated at the electrode 111 and hydrogen ions or oxygen gas may be generated at the electrode 121 by the electrolysis of water. In addition, one or more electrode reactions may alter the active agent ions depending on the kind of the active agent. If the electrolyte solution reservoir 122 contains chlorine ions, chlorine gas or hypochlorous acid may be generated.

The generation of gas in the electrode assembly 110 or 120 may inhibit energization from the electrode 111 or 121 to the active agent solution or the electrolyte solution. There is also a possibility that hydrogen ions, hydroxide ions, or hypochlorous acid generated in the electrode assembly 110 or 120 could be transferred to a biological interface and have a detrimental effect on a subject. In addition, alteration of the active agent may reduce its initial active agent effect or produce substances having an effect different to that of the active agent.

U.S. Pat. No. 4,744,787 discloses an iontophoresis device in which a silver electrode is used as an anode and a silver chloride electrode is used as a cathode.

A reaction may preferentially occur in this device, whereby silver in the anode is oxidized, forming insoluble silver chloride, while silver chloride is reduced at the cathode, forming metallic silver. These reactions may tend to suppress the generation of gas and the production of various ions due to electrode reactions as described above.

However, it may be difficult to prevent the dissolution of the silver electrode during storage of the iontophoresis device. In particular, where the device is to be used to administer a cationic active agent, usable active agents could be limited in number. In addition, large morphological changes occur upon production of silver chloride from the silver electrode. Special consideration may therefore need to be given in order to prevent such morphological changes from affecting the properties of the device. Restrictions may thus be imposed on the shape of the device (limiting use of a lamination structure, for example.)

FIG. 6 shows an iontophoresis device disclosed in JP 4-297277 A. The iontophoresis device comprises: an active electrode assembly 210 that includes an electrode 211, an electrolyte solution reservoir 212 that holds an electrolyte solution in contact with the electrode 211, an ion exchange membrane 213 of a second polarity, the ion exchange membrane 213 being placed on the outer surface of the electrolyte solution reservoir 212, an active agent solution reservoir 214 that holds an active agent solution containing active agent ions of a first polarity, the active agent solution reservoir 214 being placed on the outer surface of the ion exchange membrane 213, and an ion exchange membrane 215 of the first polarity, the ion exchange membrane 215 being placed on the outer surface of the active agent solution reservoir 214; and a counter electrode assembly 220 and an electrode 230 similar to those shown in FIG. 9.

The electrolyte solution and the active agent solution are partitioned by the second ion exchange membrane 213 of the second polarity, thus allowing the composition of the electrolyte solution to be selected independently of the active agent solution. An electrolyte solution that does not contain chlorine ions may thus be used. The selection of an electrolyte having a lower oxidation or reduction potential than the electrolysis of water as the electrolyte in the electrolyte solution may suppress the production of oxygen gas, hydrogen gas, hydrogen ions, or hydroxide ions resulting from the electrolysis of water. Furthermore, the transfer of active agent ions to the electrolyte solution reservoir may be blocked by the second ion exchange membrane, thus addressing an issue where the active agent ions may be altered due to the occurrence of an electrode reaction.

However, it may be difficult to completely separate the active agent solution in the active agent reservoir 214 and the electrolyte solution in the electrolyte solution reservoir 212.

That is, ions of the first and second polarities generated as a result of ionic dissociation of an electrolyte and undissociated electrolyte molecules generally coexistent in the electrolyte solution of the electrolyte solution reservoir 212. However, ions of the second polarity and electrolyte molecules can pass through the ion exchange membrane 213 to transfer to the active agent solution reservoir 214. Therefore, such ions or molecules may transfer to the active agent reservoir 214 and interact with the active agent ion during the storage of the device over a certain period of time, possibly reducing active agent effectiveness or causing cosmetic changes.

### BRIEF SUMMARY

In one aspect, the present disclosure is directed to an iontophoresis device capable of preventing or suppressing the generation of oxygen gas, chlorine gas, or hydrogen gas in an electrode assembly.

In another aspect, the present disclosure is directed to an iontophoresis device capable of preventing or suppressing the generation of hydrogen ions, hydroxide ions, or hypochlorous acid in an electrode.

In another aspect, the present disclosure is directed to an iontophoresis device capable of preventing or suppressing the alteration of active agent ions due to an electrode reaction upon energization.

In another aspect, the present disclosure is directed to an iontophoresis device capable of preventing or suppressing the generation of gas, ions, or the alteration of an active agent, and which causes no large changes in morphology of an electrode due to energization.

In another aspect, the present disclosure is directed to an iontophoresis device capable of preventing or suppressing the generation of gas, ions, or the alteration of an active agent, and which is capable of preventing or suppressing the alteration of active agent ions due to an electrode reaction upon energization.

In another aspect, the present disclosure is directed to an iontophoresis device capable of preventing or suppressing the generation of gas, ions, or the alteration of an active agent due to an electrode reaction upon energization, and is capable of reducing the possibility of active agent changes and/or cosmetic changes during the storage of the device.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is an explanatory view that shows a schematic configuration of an iontophoresis device.

FIGS. 2A to 2H are explanatory sectional views that show a configuration of an active electrode assembly of an iontophoresis.

FIGS. 3A to 3D are explanatory sectional views that show a configuration of a counter electrode assembly of an iontophoresis device.

FIGS. 4A to 4C are explanatory sectional views that show a configuration of an active electrode assembly of an iontophoresis device.

FIG. 5 is an explanatory view that shows a configuration of a conventional iontophoresis device.

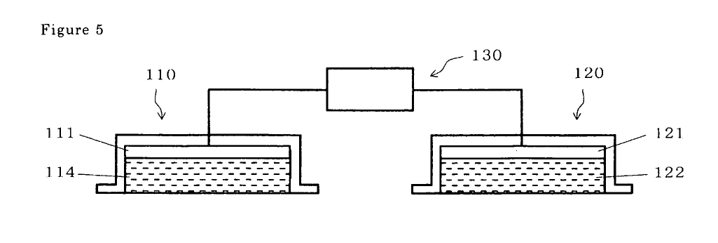

FIG. 6 is an explanatory view that shows a configuration of another conventional iontophoresis device.

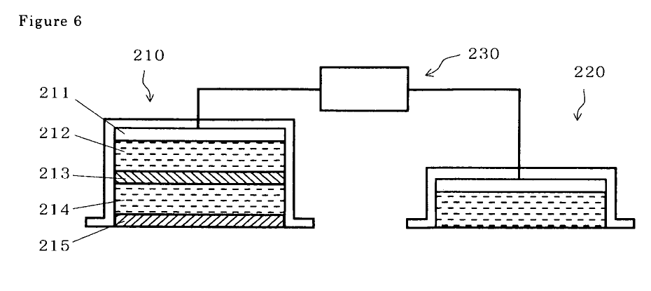

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, electric potential or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “another embodiment” means that a particular referent feature, structure, or characteristic described in connection with the embodiment is included in at least one embodiment. Thus, the appearances of the phrases “in one embodiment,” or “in an embodiment,” or “another embodiment” in various places throughout this specification are not necessarily all referring to the same embodiment. Further more, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a system for evaluating an iontophoretic active agent delivery including “a controller” includes a single controller, or two or more controllers. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein, the term bipolar membrane means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiment a gel matrix may include hydrogels, organogels, and the like. Hydrogels refer to three-dimensional network of, for example, cross-linked hydrophilic polymers in the form of a gel matrix and substantially comprising water. Hydrogels may have a net positive or negative charge, or may be neutral.

A used herein, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

A used herein, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., an active agent, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, proactive agents, metabolites, analogs, and the like. In some further embodiment, the active agent includes at least one ionic, cationic, ionizeable and/or neutral therapeutic active agent and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to positive ionsor can dissociate into a positively charged ion and a counter ion in an aqueous medium. While other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the art.

Non-limiting examples of such active agents include lidocaine, articaine, and others of the -caine class; morphine, hydromorphone, fentanyl, oxycodone, hydrocodone, buprenorphine, methadone, and similar opiod agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic active agents; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes active agents such as exenatide; as well as peptides and proteins for treatment of obesity and other maladies.

As used herein and in the claims, the term “subject” generally refers to any host, animal, vertebrate, or invertebrate, and includes fish, mammals, amphibians, reptiles, birds, and particularly humans.

The term “ionic liquid” as used herein refers to a molten salt present as a liquid at or near room temperature. An anion comprising an ionic liquid may be selected from PF6-, BF4-, AlCl4-, ClO4-, a hydrogen sulfate ion represented by the following formula (1), bis-trifluoro-alkylsulfonyl-imide represented by the following formula (2), trifluoro-methane sulfonate represented by the following formula (3), or a combination thereof. 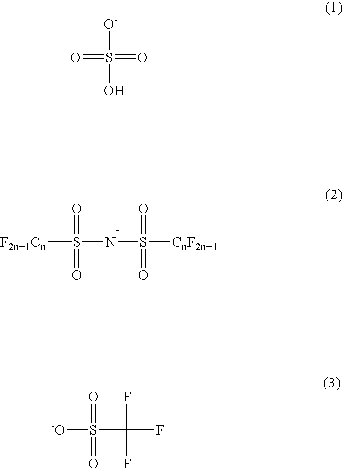
It should be noted that “n” in the formula (2) represents a positive integer.

A cation comprising an ionic liquid may be selected from: an imidazolium derivative containing monoalkylimidazolium represented by the following formula (4), dialkylimidazolium represented by the following formula (5), or trialkylimidazolium represented by the following formula (6); a pyridinium derivative containing 1-alkylpyridinium represented by the following formula (7); a piperidinium derivative containing dialkylpiperidinium represented by the following formula (8); a pyrolidinium derivative containing 1-alkylpyrolidinium represented by the following formula (9); a tetra-alkyl ammonium derivative containing tetra-alkyl ammonium represented by the following formula (10); or a combination thereof. 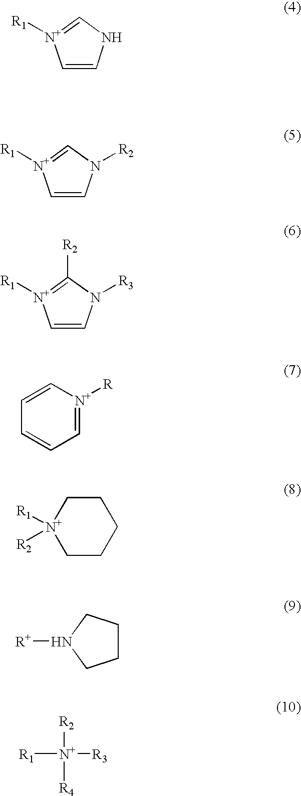
It should be noted that R and R1 to R4 in the formulas (4) to (10) each represent an arbitrary alkyl or fluoroalkyl group.

The headings provided herein are for convenience only and do not interpret the scope or meaning of the embodiments.

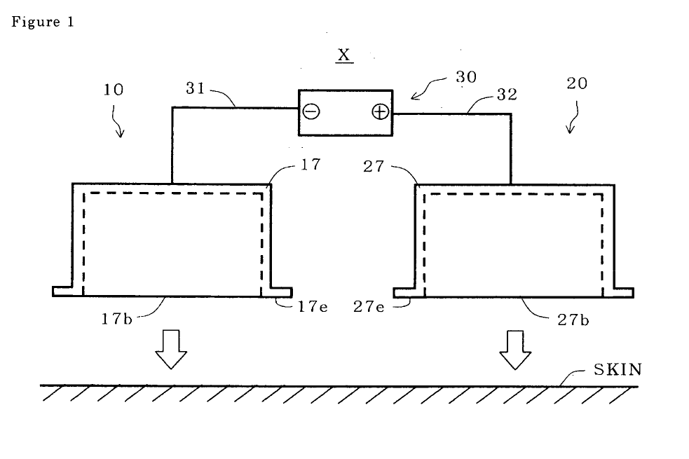

FIG. 1 is an explanatory view showing the schematic configuration of an iontophoresis device X.

The iontophoresis device X comprises: an electric power source 30; an active electrode assembly 10 coupled to the positive pole of the electric power source 30 using an electric supply line 31; and a counter electrode assembly 20 coupled to the negative pole of the electric power source 30 using an electric supply line 32.

The active electrode assembly 10 includes a container 17, and the counter electrode assembly 20 includes a container 27. The containers 17 and 27 each include a space capable of housing various structures to be described later.

The containers 17 and 27 may be formed by using any variety of materials such as a plastic. It may be effective to employ a flexible material capable of preventing the evaporation of water from the inside of the container and the ingress of foreign matter from the outside, and capable of conforming to the movement of a subject or the irregularities of a biological interface of the subject. In addition, a lower portion 17b of the container 17 and a lower portion 27b of the container 27 may be open, and a removable liner of an appropriate material for preventing the evaporation of water and the mixing of foreign matter during storage of the iontophoresis device X may be attached to the lower portion 17b of the container 17 or the lower portion 27b of the container 27. An adhesive layer for improving adhesiveness to a biological interface upon administration of an active agent may be placed on a lower end portion 17e of the container 17 or a lower end portion 27e of the container 27.

A battery, a constant electric potential device, a constant current device, a constant electric potential/current device, or the like may be used as the electric power source 30.

The iontophoresis device X may administer active agent ions to a subject through energization from the electric power source 30 in a state where the lower portions 17b and 27b of the active electrode assembly 10 and the counter electrode assembly 20 are brought into contact with a biological interface of the subject.

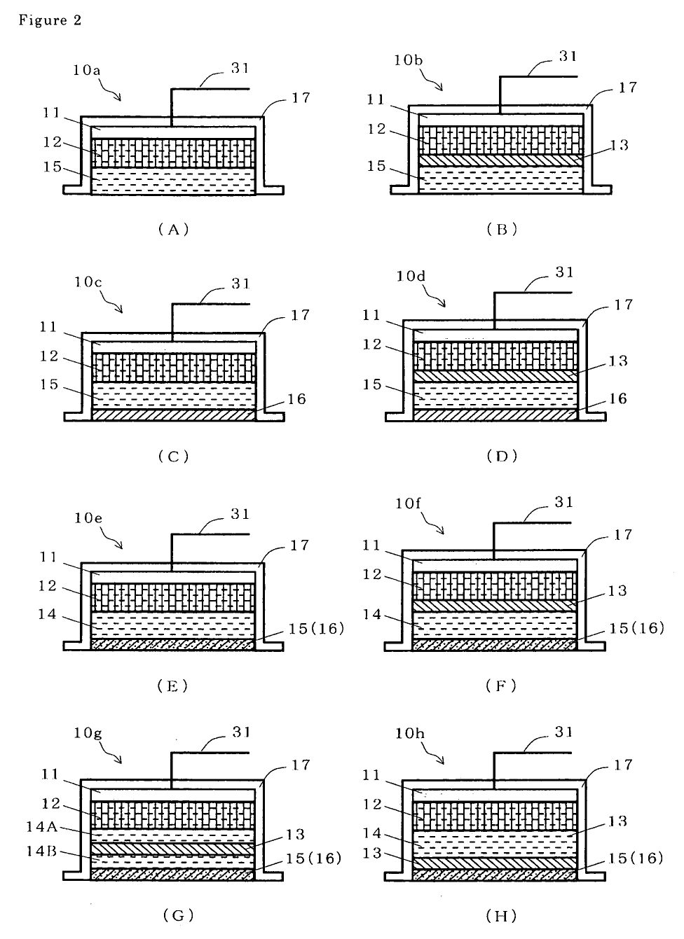

FIGS. 2A to 2H are explanatory sectional views showing configurations of active electrode assemblies 10a to 10h, respectively, any of which may be used as the active electrode assembly 10 of the iontophoresis device X.

The active electrode assembly 10a of FIG. 2A comprises: an electrode 11 connected to the electric supply line 31 of the electric power source 30; an ionic liquid reservoir 12 that holds an ionic liquid in contact with the electrode 11; and an active agent reservoir 15 that holds an active agent solution, the active agent reservoir 15 being arranged on the outer surface of the ionic liquid reservoir 12.

An electrode comprising an arbitrary conductive material may be used for the electrode 11 without any particular limitation. It may be preferable to use an inactive electrode material such as gold, platinum, carbon, or the like rather than an active electrode material such as silver or the like in order to avoid changes in morphology of the electrode 11.

The ionic liquid of the ionic liquid reservoir 12 is a salt molten at normal temperature, comprising: an anion selected from PF6-, BF4-, AlCl4-, ClO4-, a hydrogen sulfate ion, bis-trifluoro-alkylsulfonyl-imide, trifluoro-methane sulfonate, or a combination thereof; and a cation selected from an imidazolium derivative, a pyridinium derivative, a piperidinium derivative, a pyrolidinium derivative, a tetra-alkyl ammonium derivative, or a combination thereof.

When bis-trifluoro-alkylsulfonyl-imide is selected as the anion of the ionic liquid, hydrophobicity can be imparted to the ionic liquid. Therefore, separability between the ionic liquid of the ionic liquid reservoir 12 and the active agent solution of the active agent reservoir 15 can be improved.

In addition, the above ionic liquid may be blended with an electrolyte having a lower oxidation potential than that of the ionic liquid. Blending may reduce an electric potential necessary to cause energization from the electrode 11 to the ionic liquid reservoir 12.

Examples of electrolytes that may be used include: ferrous sulfate; ferric sulfate; ascorbic acid; sodium ascorbate; and lactic acid, oxalic acid, malic acid, succinic acid, and fumaric acid, or salts thereof.

The ionic liquid reservoir 12 may hold the ionic liquid in a liquid state. Alternatively, the portion may hold the ionic liquid in a state where an appropriate absorbing carrier (such as a microporous body or a sponge-like polymer (for example, a polyimide porous membrane or a poly-tetrafluoro-ethylene microporous membrane)) is impregnated with the ionic liquid. Separability between the ionic liquid of the ionic liquid reservoir 12 and the active agent solution of the active agent reservoir 15 may be improved in this case.

The active agent solution of the active agent reservoir 15 may be a solution of an active agent whose active agent component dissociates into positive active agent ions. The active agent reservoir 15 can hold the active agent solution in a liquid state. Alternatively, when the portion holds the active agent solution with which an appropriate absorbing carrier such as gauze, filter paper, or a gel matrix is impregnated, separability between the ionic liquid of the ionic liquid reservoir 12 and the active agent solution of the active agent reservoir 15 may be improved.

In the active electrode assembly 10a, active agent ions in the active agent reservoir 15 may be administered to a subject by applying a positive electric potential to the electrode 11 in a state where the active agent reservoir 15 is brought into contact with a biological interface of a subject. Energization from the electrode 11 to the ionic liquid reservoir 12 in this case may be caused by the oxidation of an anion or cation comprising the ionic liquid. Alternatively, when the ionic liquid is blended with an electrolyte having a lower oxidation potential than that of the ionic liquid, energization from the electrode 11 to the ionic liquid reservoir 12 may be caused by the oxidation of the electrolyte. Accordingly, the generation of oxygen gas or chlorine gas, and the production of hydrogen ions or hypochlorous acid due to energization may be suppressed.

Energization from the ionic liquid reservoir 12 to the active agent reservoir 15 is mainly caused by the transfer of an active agent counter ion in the active agent reservoir 15 to the ionic liquid reservoir 12.

Furthermore, a cation comprising the ionic liquid tends to not transfer to the active agent reservoir 15 due to energization from the ionic liquid reservoir 12 to the active agent reservoir 15 because the cations described above that may comprise the ionic liquid are hydrophobic. Accordingly, alteration of active agent ions and the transfer of the cations comprising the ionic liquid to the active agent reservoir 15 may be avoided.

In an iontophoresis device that administers an active agent whose active agent component dissociates into negative active agent ions, bis-trifluoro-alkylsulfonyl-imide may be selected to comprise the ionic liquid in order to suppress or prevent the transfer of anions comprising the ionic liquid to the active agent reservoir 15 upon energization.

The active electrode assembly 10b of FIG. 2B comprises: the electrode 11, the ionic liquid reservoir 12, and the active agent reservoir 15 similar to those of the active electrode assembly 10a; and the anion exchange membrane 13 between the ionic liquid reservoir 12 and the active agent reservoir 15.

The active electrode assembly 10b is similar to the active electrode assembly 10a. In addition, the anion exchange membrane 13 may block the transfer of active agent ions to the ionic liquid reservoir 12 and the transfer of positive ions in the ionic liquid reservoir 12 (a cation comprising the ionic liquid and positive ions generated by the dissociation of an electrolyte with which the ionic liquid is blended) to the active agent reservoir 15.

The alteration of the active agent ions due to an electrode reaction may thus be suppressed or prevented. Further, the alteration of the active agent ions or a reduction in safety to a subject due to the positive ions that have transferred from the ionic liquid reservoir 12 to the active agent reservoir 15 may be suppressed or prevented.

Use of an anion exchange membrane having as high a transport number as possible may be preferably used. An anion exchange membrane prepared by filling the pores of a porous film with an anion exchange resin may also be preferable.

The active electrode assembly 10c of FIG. 2C comprises: the electrode 11, the ionic liquid reservoir 12, and the active agent reservoir 15 similar to those of the active electrode assembly 10a; and a cation exchange membrane 16 on the outer surface of the active agent reservoir 15.

The active electrode assembly 10c is similar to the active electrode assembly 10a. In addition, the active electrode assembly 10c may increase the transport number for active agent ions upon administration of an active agent because the cation exchange membrane 16 can block the transfer of biological counter ions from a subject to the active agent reservoir 15.

The active electrode assembly 10d of FIG. 2D comprises: the electrode 11, the ionic liquid reservoir 12, the anion exchange membrane 13, and the active agent reservoir 15 similar to those of the active electrode assembly 10b; and a cation exchange membrane 16 on the outer surface of the active agent reservoir 15.

The active electrode assembly 10d is similar to the active electrode assembly 10b. In addition, the active electrode assembly 10d may increase the transport number of active agent ions upon administration of an active agent because the cation exchange membrane 16 can block the transfer of a biological counter ion from a subject to the active agent reservoir 15.

In each of the active electrode assemblies 10c and 10d, a cation exchange membrane having as high a transport number as possible is preferably used for the cation exchange membrane 16 for improving an increasing effect on the transport number of an active agent ion. A cation exchange membrane prepared by filling the pores of a porous film with a cation exchange resin may be preferable.

The anion exchange membrane 13 in each of the active electrode assemblies 10b and 10d may be replaced by using a membrane filter capable of substantially blocking the passage of active agent ions and/or positive ions in the ionic liquid reservoir 12 while substantially permitting the passage of active agent counter ions.

The active electrode assembly 10e of FIG. 2E comprises: the electrode 11 and the ionic liquid reservoir 12 similar to those of the active electrode assembly 10a; an electrolyte solution reservoir 14 that holds an electrolyte solution, the electrolyte solution reservoir 14 being arranged on the outer surface of the ionic liquid reservoir 12; and the active agent reservoir 15 comprising the cation exchange membrane 16 doped with an active agent ion, the active agent reservoir 15 being arranged on the outer surface of the electrolyte solution reservoir 14.

The electrolyte solution reservoir 14 may hold an arbitrary electrolyte solution to ensure a conductive path from the ionic liquid reservoir 12 to the active agent reservoir 15. However, use of an electrolyte solution free of any positive ions having a mobility comparable to, or lower than, that of active agent ions may further increase the transport number of the active agent ions upon energization.

The electrolyte solution reservoir 14 may hold the electrolyte solution in a liquid state. Alternatively, when the portion holds the electrolyte solution with which an appropriate absorbing carrier such as gauze, filter paper, or a gel matrix is impregnated, separability between the ionic liquid of the ionic liquid reservoir 12 and the electrolyte solution of the electrolyte solution reservoir 14 may improve.

Cation exchange membranes similar to those used in each of the active electrode assemblies 10c and 10d may also be used for the cation exchange membrane 16. The cation exchange membrane 16 may be doped with active agent ions by immersing the cation exchange membrane 16 in an active agent solution having an appropriate concentration. The amount of active agent ions with which the cation exchange membrane 16 is doped can be adjusted depending on, for example, the concentration of an active agent solution used, an immersion time period, and the number of immersions. The active agent ions are thought to bind to cation exchange groups in the cation exchange membrane 16 through ionic bonds when the cation exchange membrane 16 is doped with active agent ions.

Energization from the electrode 11 to the ionic liquid reservoir 12 in the active electrode assembly 10e may occur in a manner similar to that of the active electrode assembly 10a. Therefore, the generation of oxygen gas, chloride gas, and the production of hydrogen ions or hypochlorous acid due to energization can be suppressed.

Energization from the ionic liquid reservoir 12 to the electrolyte solution reservoir 14 is mainly due to the transfer of negative ions in the electrolyte solution reservoir 14 to the ionic liquid reservoir 12. Energization from the electrolyte solution reservoir 14 to the active agent reservoir 15 is due to the transfer of positive ions in the electrolyte solution reservoir 14 to the active agent reservoir 15. Without being limited by theory, it is believed that active agent ions used to dope the cation exchange membrane 16 of the active agent reservoir 15 are replaced by positive ions from the electrolyte solution reservoir 14, and thus administered to a subject.

The efficiency of the administration of active agent ions may increase with the active electrode assembly 10e because the cation exchange membrane 16 can block the transfer of a biological counter ion to the active agent reservoir 15.

The efficiency of the administration of the active agent ions may additionally be increased with the active electrode assembly 10e because the administration of the active agent ions is performed in a state where the cation exchange membrane 16 doped with the active agent ions is brought into direct contact with a biological interface of a subject.

The stability of active agent ions during storage may increase with the active electrode assembly 10e, and a reduction in the amount of stabilizers, antibacterial agents, antiseptics, and the like may be achieved because the active agent ions may be held doped in the cation exchange membrane 16.

The active electrode assembly 10f of FIG. 2F comprises: the electrode 11, the ionic liquid reservoir 12, the electrolyte solution reservoir 14, and the active agent reservoir 15 similar to those of the active electrode assembly 10e; and the anion exchange membrane 13 between the ionic liquid reservoir 12 and the electrolyte solution reservoir 14.

An anion exchange membrane similar to that described above with respect to the active electrode assembly 10b may be used for the anion exchange membrane 13.

The active electrode assembly 10f is similar to the active electrode assembly 10e. Further, the movement of positive ions between the ionic liquid reservoir 12 and the electrolyte solution reservoir 14 may be suppressed or blocked.

The alteration of active agent ions in the cation exchange membrane 16 due to an electrode reaction upon energization may thus be suppressed or prevented because the transfer of the active agent ions to the ionic liquid reservoir 12 via the electrolyte solution reservoir 14 can be prevented.

The alteration of active agent ions and a reduction in safety may also be suppressed or prevented because the transfer of positive ions in the ionic liquid reservoir 12 to the active agent reservoir 15 via the electrolyte solution reservoir 14 can be prevented.

The anion exchange membrane 13 in the active electrode assembly 10f can be replaced by using a membrane filter capable of substantially blocking the passage of positive ions in the ionic liquid reservoir 12 (particularly cations comprising the ionic liquid) while substantially permitting the passage of negative ions in the electrolyte solution reservoir 14.

The active electrode assembly 10g of FIG. 2G differs from the active electrode assembly 10f only in that: two electrolyte solution reservoirs 14A and 14B are arranged between the ionic liquid reservoir 12 and the active agent reservoir 15; and the anion exchange membrane 13 is arranged between the two electrolyte solution reservoirs 14A and 14B. The active electrode assembly 10g is otherwise similar to the active electrode assembly 10f in structure and effect.

The active electrode assembly 10h of FIG. 2H comprises: the electrode 11, the ionic liquid reservoir 12, the electrolyte solution reservoir 14, and the active agent reservoir 15 similar to those of the active electrode assembly 10e; and the anion exchange membrane 13 between the electrolyte solution reservoir 14 and the active agent reservoir 15.

An anion exchange membrane having a relatively low transport number (for example, a transport number from 0.7 to 0.98) may be used for the anion exchange membrane 13 in the active electrode assembly 10h.

Energization from the electrode 11 to the ionic liquid reservoir 12 and energization from the ionic liquid reservoir 12 to the electrolyte solution reservoir 14 in the active electrode assembly 10h each occur in a manner similar to that described above with respect to the active electrode assembly 10e.

Energization from the electrolyte solution reservoir 14 to the active agent reservoir 15 is caused by the transfer of positive ions in the electrolyte solution reservoir 14, which has passed through the anion exchange membrane 13, to the active agent reservoir 15. Without limitation to theory, active agent ions with which the cation exchange membrane 16 of the active agent reservoir 15 is doped are substituted by positive ions from the electrolyte solution reservoir 14, and thus transferred to a subject.

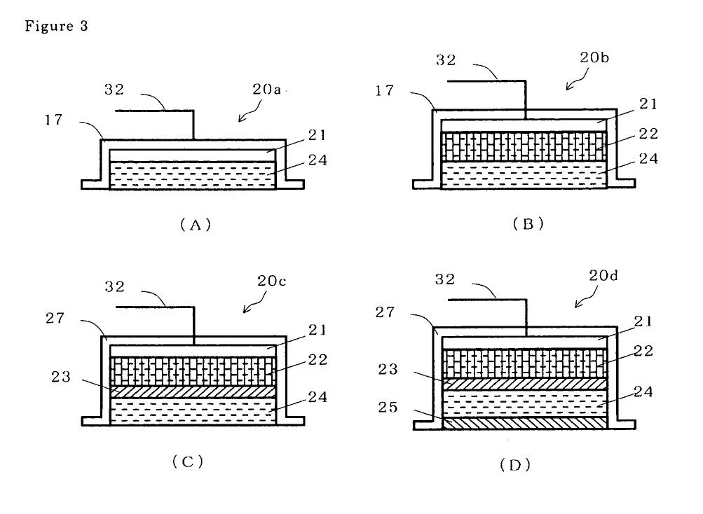

FIGS. 3A to 3D are explanatory sectional views showing configurations of counter electrode assemblies 20a to 20d, respectively, each of which can be used as the counter electrode assembly 20 of the iontophoresis device X.

The counter electrode assembly 20a of FIG. 3A comprises: the electrode 21 connected to an electric supply line 32; an electrolyte solution reservoir 24 that holds an electrolyte solution in contact with the electrode 21.

The use of an active electrode comprising silver chloride or the like for the electrode 21 may prevent the generation of hydrogen gas or hydroxyl ions due to the electrolysis of water. An inactive conductive electrode material such as gold, platinum, carbon, or the like may also be used when an electrolyte solution prepared by dissolving an electrolyte having a lower reduction potential than that of water is used as the electrolyte solution of the electrolyte solution reservoir 24.

The electrolyte solution reservoir 24 may hold an any of a variety of electrolyte solutions that ensure energization from the electrode 21 to a subject. When an electrolyte solution prepared by dissolving an electrolyte having a lower reduction potential than that of water or a buffer electrolyte solution prepared by dissolving multiple kinds of electrolytes is used, the generation of hydrogen gas due to an electrode reaction and a fluctuation in pH due to the production of hydrogen ions may be prevented.

Examples of electrolytes which may be used include: inorganic compounds such as ferrous sulfate and ferric sulfate; active agents such as ascorbic acid and sodium ascorbate; acidic compounds each present on the surface of a biological interface such as lactic acid; and organic acids such as oxalic acid, malic acid, succinic acid, and fumaric acid and/or salts thereof.

The electrolyte solution reservoir 24 may hold the electrolyte solution in a liquid state. Alternatively, when the portion holds the electrolyte solution with which an appropriate absorbing carrier such as gauze, filter paper, or a gel matrix is impregnated, the handleability of the electrolyte solution may be improved.

The counter electrode assembly 20a may serve as a counter electrode of the active electrode assembly 10. The counter electrode assembly 20a closes a current path ranging from the positive pole of the electric power source 30 to the negative pole of the electric power source 30 via the active electrode assembly 10, a subject, and the counter electrode assembly 20a.

The counter electrode assembly 20b of FIG. 3B comprises: the electrode 21 connected to an electric supply line 32; an ionic liquid reservoir 22 that holds an ionic liquid in contact with the electrode 21; and the electrolyte solution reservoir 24 arranged on the outer surface of the ionic liquid reservoir 22.

An electrode comprising an arbitrary conductive material can be used for the electrode 21 of the counter electrode assembly 20b, without any particular limitations. It may be preferable to use an inactive electrode material such as gold, platinum, carbon, or the like rather than an active electrode material such as silver chloride or the like in order to avoid changes in morphology of the electrode 21.

The ionic liquid reservoir 22 may be configured in a manner similar to that of the ionic liquid reservoir 12.

The electrolyte solution reservoir 24 may hold an electrolyte solution for securing energization property from the ionic liquid reservoir 12 to a subject, and may hold any of a variety of electrolyte solutions such as a saline.

The electrolyte solution reservoir 24 can hold the electrolyte solution in a liquid state. Alternatively, when the electrolyte solution is held in an appropriate absorbent carrier such as gauze, filter paper, or a gel matrix, separability between the ionic liquid of the ionic liquid reservoir 22 and the electrolyte solution of the electrolyte solution reservoir 24 may be improved.

In the counter electrode assembly 20b, energization from the electrode 21 to the ionic liquid reservoir 22 may be caused by the reduction of anions or cations comprising the ionic liquid. Alternatively, energization may be caused by the reduction of the electrolyte when the ionic liquid is blended with an electrolyte having a lower reduction potential than that of the ionic liquid.

Accordingly, the counter electrode assembly 20b is similar to the counter electrode assembly 20a. In addition, the production of hydrogen gas and hydroxyl ions may also be suppressed.

The counter electrode assembly 20c of FIG. 3C comprises: the electrode 21, the ionic liquid reservoir 22 and the electrolyte solution reservoir 24 similar to those of the counter electrode assembly 20b; and the cation exchange membrane 23 being placed between the ionic liquid reservoir 22 and the electrolyte solution reservoir 24.

The counter electrode assembly 20c is similar to the counter electrode assembly 20b. In addition, the cation exchange membrane 23 may substantially block the transfer of negative ions from the ionic liquid reservoir 22 to the electrolyte solution reservoir 24.

A cation exchange membrane having as high a transport number as possible may be preferably used for the cation exchange membrane 23. A cation exchange membrane prepared by filling the pores of a porous film with a cation exchange resin may be used.

The counter electrode assembly 20d of FIG. 3D comprises: the electrode 21, the ionic liquid reservoir 22, the cation exchange membrane 23 and the electrolyte solution reservoir 24 similar to those of the counter electrode assembly 20c; and the anion exchange membrane 25 being placed on the outer surface of the electrolyte solution reservoir 24.

The counter electrode assembly 20d is similar to the counter electrode assembly 20c. In addition, an ion balance at an interface between the anion exchange membrane 25 and a biological interface may be better maintained because the anion exchange membrane 25 is arranged on the outer surface of the electrolyte solution reservoir 24.

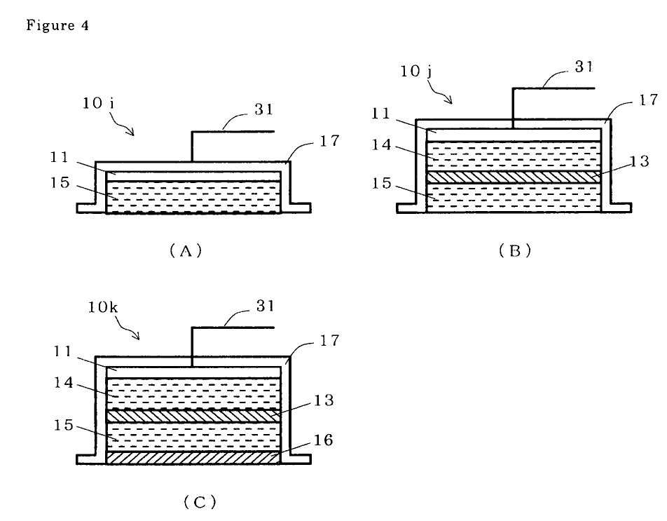

FIGS. 4A to 4C are explanatory sectional views showing configurations of active electrode assemblies 10i to 10k, respectively, each of which may be used as the active electrode assembly 10 of the iontophoresis device X. Each of the active electrode assemblies 10i to 10k may be combined with the counter electrode assembly 20b, 20c, or 20d to configure the iontophoresis device X.

The active electrode assembly 10i of FIG. 4A comprises: the electrode 11 connected to the electric supply line 31 of the electric power source 30; and the active agent reservoir 15 that holds an active agent solution in contact with the electrode 11.

The active agent reservoir 15 of the active electrode assembly 10i may be configured in a manner similar to that of the active agent reservoir 15 of the active electrode assembly 10a. A silver electrode may be used for the electrode 11 to substantially prevent the generation of oxygen gas or chlorine gas due to an electrode reaction, and substantially prevent the production of hydrogen ions.

The active electrode assembly 10j of FIG. 4B comprises: the electrode 11 connected to the electric supply line 31 of the electric power source 30; the electrolyte solution reservoir 14 that holds an electrolyte solution in contact with the electrode 11; the anion exchange membrane 13 arranged on the outer surface of the electrolyte solution reservoir 14; and the active agent reservoir 15 that holds an active agent solution, the active agent reservoir 15 being arranged on the outer surface of the anion exchange membrane 13.

The active agent reservoir 15 in the active electrode assembly 10j can be configured in a manner similar to that of the active agent reservoir of the active electrode assembly 10a. An anion exchange membrane similar to that described above with respect to the active electrode assembly 10b may be used for the anion exchange membrane 13 of the active electrode assembly 10j.

An electrolyte solution prepared by dissolving an electrolyte having a lower oxidation potential than that of water, or a buffer electrolyte solution prepared by dissolving multiple kinds of electrolytes, may be used as the electrolyte solution of the electrolyte solution reservoir 14 in the active electrode assembly 10j. In this case, the generation of hydrogen gas or hydrogen ions due to an electrode reaction may be substantially prevented even if an inactive electrode comprising gold, platinum, carbon, or the like is used for the electrode 11.

The active electrode assembly 10j is similar to the active electrode assembly 10i. In addition, the active electrode assembly 10j may substantially prevent the alteration of active agent ions due to an electrode reaction upon energization because the anion exchange membrane 13 can block the transfer of the active agent ions from the active agent reservoir 15 to the electrolyte solution reservoir 14.

The active electrode assembly 10k of FIG. 4C comprises: the electrode 11, the electrolyte solution reservoir 14, the anion exchange membrane 13, and the active agent reservoir 15 similar to those of the active electrode assembly 10j; and the cation exchange membrane 16 arranged on the outer surface of the active agent reservoir 15.

A cation exchange membrane similar to that described above with respect to the active electrode assembly 10c may be used for the cation exchange membrane 16.

The active electrode assembly 10k is similar to the active electrode assembly 10j. In addition, an increase in transport number of active agent ions may be achieved because the cation exchange membrane 16 can block the transfer of a biological counter ion from the side of a subject to the active agent reservoir 15.

The above description of illustrated embodiments, including what is described in the Abstract, is not intended to be exhaustive or to limit the embodiments to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein of the various embodiments can be applied to other problem-solving systems devices, and methods, not necessarily the exemplary problem-solving systems devices, and methods generally described above.

Further, although a single active electrode assembly and a single counter electrode assembly connected to an electric power source are described above, multiple active electrode assemblies and/or multiple counter electrode assemblies may also be employed.

Also, the iontophoresis device need not be provided with a counter electrode assembly. An active agent may be administered by bringing an active electrode assembly into contact with a biological interface of a subject; and applying an electric potential to the active electrode assembly in a state where a portion of the subject is brought into contact with a member to serve as ground.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety.

Aspects of the embodiments can be modified, if necessary, to employ systems, circuits, and concepts of the various patents, applications, and publications to provide yet further embodiments.

All of the above U.S. patents, U.S. patent application publications, U.S. patent applications, foreign patents, foreign patent applications and non-patent publications referred to in this specification and/or listed in the Application Data Sheet, including but not limited to U.S. Provisional Application No. 60/726,803, filed Oct. 14, 2005; and Japanese Application No. 2005-266623, filed Sep. 14, 2005, are incorporated herein by reference, in their entirety.

These and other changes can be made to the embodiments in light of the above-detailed description. In general, in the following claims, the terms used should not be construed to limit the invention to the specific embodiments disclosed in the specification and the claims, but should be construed to include all possible embodiments along with the full scope of equivalents to which such claims are entitled. Accordingly, the scope of the invention shall only be construed and defined by the scope of the appended claims.

What is claimed is:

1. An iontophoresis device, comprising:
an active electrode assembly comprising:
a first electrode supplied with an electric potential of a first polarity;
an ionic liquid reservoir that holds an ionic liquid in contact with the first electrode; and
an active agent reservoir placed on an outer surface of the ionic liquid reservoir, the active agent reservoir holding active agent ions of the first polarity.

2. The iontophoresis device according to claim 1, wherein the active electrode assembly further comprises an ion exchange membrane of the first polarity on an outer surface of the active agent reservoir.

3. The iontophoresis device according to claim 1, wherein:
the active agent reservoir holds an active agent solution that comprises the active agent ions and active agent counter ions of a second polarity; and
the active electrode assembly further comprises a semi-permeable membrane that selectively allows passage of at least the active agent counter ions between the active agent reservoir and the ionic liquid reservoir.

4. The iontophoresis device according to claim 1, wherein an electrolyte having an oxidation-reduction potential lower than that of the ionic liquid is dissolved in the ionic liquid.

5. The iontophoresis device according to claim 1, wherein the ionic liquid comprises an imidazorium derivative, a pyridinium derivative, a piperidinium derivative, a pyrrolidinium derivative, or a tetra-alkyl-ammonium derivative.

6. The iontophoresis device according to claim 1, wherein the ionic liquid is hydrophobic.

7. The iontophoresis device according to claim 6, wherein the active electrode assembly further comprises an ion exchange membrane of the first polarity on an outer surface of the active agent reservoir.

8. The iontophoresis device according to claim 6, wherein:
the active agent reservoir holds an active agent solution that comprises the active agent ions and active agent counter ions of a second polarity; and
the active electrode assembly further comprises a semi-permeable membrane that selectively allows passage of at least the active agent counter ions between the active agent reservoir and the ionic liquid reservoir.

9. The iontophoresis device according to claim 6, wherein an electrolyte having an oxidation-reduction potential lower than that of the ionic liquid is dissolved in the ionic liquid.

10. The iontophoresis device according to claim 6, wherein the ionic liquid comprises an imidazorium derivative, a pyridinium derivative, a piperidinium derivative, a pyrrolidinium derivative, or a tetra-alkyl-ammonium derivative.

11. The iontophoresis device according to claim 6, wherein the ionic liquid comprises bis-trifluoroalkyl-sulphonyl-imide.

12. The iontophoresis device according to claim 11, wherein the active electrode assembly further comprises an ion exchange membrane of the first polarity on an outer surface of the active agent reservoir.

13. The iontophoresis device according to claim 11, wherein:
the active agent reservoir holds an active agent solution that comprises the active agent ions and active agent counter ions of a second polarity; and
the active electrode assembly further comprises a semi-permeable membrane that selectively allows passage of at least the active agent counter ions between the active agent reservoir and the ionic liquid reservoir.

14. The iontophoresis device according to claim 11, wherein an electrolyte having an oxidation-reduction potential lower than that of the ionic liquid is dissolved in the ionic liquid.

15. The iontophoresis device according to claim 11, wherein the ionic liquid comprises an imidazorium derivative, a pyridinium derivative, a piperidinium derivative, a pyrrolidinium derivative, or a tetra-alkyl-ammonium derivative.

16. The iontophoresis device according to claim 15, wherein the active electrode assembly further comprises an ion exchange membrane of the first polarity on an outer surface of the active agent reservoir.

17. The iontophoresis device according to claim 15, wherein:
the active agent reservoir holds an active agent solution that comprises the active agent ions and active agent counter ions of a second polarity; and
the active electrode assembly further comprises a semi-permeable membrane that selectively allows passage of at least the active agent counter ions between the active agent reservoir and the ionic liquid reservoir.

18. The iontophoresis device according to claim 15, wherein an electrolyte having an oxidation-reduction potential lower than that of the ionic liquid is dissolved in the ionic liquid.

19. The iontophoresis device according to claim 18, wherein the active electrode assembly further comprises an ion exchange membrane of the first polarity on an outer surface of the active agent reservoir.

20. The iontophoresis device according to claim 18, wherein:
the active agent reservoir holds an active agent solution that comprises the active agent ions and active agent counter ions of a second polarity; and
the active electrode assembly further comprises a semi-permeable membrane that selectively allows passage of at least the active agent counter ions between the active agent reservoir and the ionic liquid reservoir.

21. The iontophoresis device according to claim 20, wherein the active electrode assembly further comprises an ion exchange membrane of the first polarity on an outer surface of the active agent reservoir.

22. The iontophoresis device according to claim 1, wherein:
the active agent reservoir comprises an ion exchange membrane of the first polarity doped with the active agent ions;
the active electrode assembly further comprises an electrolyte solution reservoir that holds an electrolyte solution, the electrolyte solution reservoir placed on an outer surface of the ionic liquid reservoir, and a semi-permeable membrane that selectively allows passage of at least ions of a second polarity of the electrolyte solution reservoir, the semi-permeable membrane placed between the ionic liquid reservoir and the active agent reservoir; and
the active agent reservoir is placed on an outer surface of the electrolyte solution reservoir.

23. The iontophoresis device according to claim 1, further comprising:
a counter electrode assembly comprising:
a second electrode supplied with an electric potential of the second polarity;
a second ionic liquid reservoir that holds an ionic liquid in contact with the second electrode; and
a second electrolyte solution reservoir that holds an electrolyte solution, the second electrolyte solution reservoir placed on an outer surface of the second ionic liquid reservoir.

24. An iontophoresis device comprising:
an active electrode assembly comprising:
a first electrode; and
an active agent reservoir that holds active agent ions of a first polarity, the active agent ions being administered to a living body by an electric potential of a first polarity applied to the first electrode; and
a counter electrode assembly comprising:
a second electrode supplied with an electric potential of a second polarity;
a second ionic liquid reservoir that holds an ionic liquid in contact with the second electrode; and
an electrolyte solution reservoir that holds an electrolyte solution, the electrolyte solution reservoir placed on an outer surface of the second ionic liquid reservoir.
